(19) United States
(12) Patent Application Publication
(10) Pub. No.: US 2009/0306749 A1
(43) Pub. Date: Dec. 10, 2009

(54) Therapeutic hot and cold water belt

(76) Inventor: Damalie Mulindwa, San Pedro, CA (US)

Correspondence Address: Damalie Mulindwa, 1180 W. 7th Street # 2, San Pedro, CA, 90731, US

(21) Appl. No.: 12/455,746

(22) Filed: Jun. 5, 2009

Related U.S. Application Data
(60) Provisional application No. 61/131,224, filed on Jun. 7, 2008.

(51) Int. Cl. A61F 7/00; B65D 25/14

(52) U.S. Cl. 607/108; 607/112; 220/495.06

(57) ABSTRACT

The therapeutic hot and cold water belt (20), which offers comfortable relief from pain, is placed about the waist, lower abdomen, back or any other part of the body, and then secured in place by an elasticized belt terminating in a hook-and-loop fastener system. The belt includes a water pouch (32) to receive and contain water. Ribs (46, 48, 50) are secured to the inside of the pouch, which divide it into compartments (46a, 46b, 46c, 46d), and maintain the shape of the pouch so that it will not balloon out when the water is inserted therein. The ribs also limit and thereby conserve the amount of water placed in the pouch. For comfort against the body, the pouch may be covered with a combed cotton material (54), which is secured also by a hook-and-loop fastener system. A funnel opening (34) to the pouch allows easy filling of the pouch with water. The funnel opening is secured with a screw (42) to prevent any pressure on the pouch from forcing the water flow out. Another cover (44) folds back from the top of the plastic pouch container to prevent the screw plug from having contact with the wearer. The user determines the temperature of water poured inside, whether hot, cold or warm. The system is placed on the body, and positioned and secured in place as desired.

### CROSS-REFERENCE TO RELATED APPLICATION

This application claims the benefit of U.S. Provisional Application No. 61/131,224, filed 7 Jun. 2008.

### REFERENCE REGARDING FEDERAL SPONSORSHIP

Not Applicable

### REFERENCE TO MICROFICHE APPENDIX

Not Applicable

### BACKGROUND OF THE INVENTION

1. Field of the Invention

The present invention relates to therapeutic belts into which water, at any desired temperature, can be inserted for where any degree of heating or cooling is needed or sought. Such belts are placeable on or about any part of the wearer's body, and are particularly useful by women for relief of menstrual cramps and after-birth pain as well as for other stomach and back pains. However, such belts are may also be used by men and youth.

2. Description of Related Art and Other Considerations

Many people suffer from stomach, lower back and pain on other parts of the body. Women often experience discomfort during their menstrual cycle. It is common for some consumers to apply a warm compress or pad to the afflicted area of the body. However, many such compresses and pads will not remain on the desired area upon movement of the wearer and slip or otherwise move away from where first placed, which might limit relief and, at the very least, is frustrating.

### SUMMARY OF THE INVENTION

These and other problems are successfully addressed and overcome by the therapeutic hot and cold water belt, which comprises a strong stretchy elasticized belt supporting an attached pouch for containing water. Ribs with through openings or holes are secured on the inside of the pouch and divide the pouch into compartments, the rib openings permitting water to flow into the compartments. Importantly, the ribs maintain the shape of the pouch (preferably configured to be thin and wide and having an ovally formed container) and prevent it from ballooning out when water is inserted inside the pouch. For comfort against the body, the pliable pouch container is covered with a combed cotton material, which is secured with hook and loop fasteners at the top. A funnel, terminating in an internally threaded opening to the pouch, is located at its top and allows the user to pour water into the pouch. A threaded hard plastic plug is screwed into the spout entry so that any pressure applied on the pouch will not permit the water to flow out. Another cover folds back from the top of the pouch to cover the plug and to prevent its contact with the wearer. The user determines the temperature of water poured inside the pouch, whether hot, warm or cold. The system is placeable on the body, and positioned as desired, on the wearer's front or back or any other part of the body. It is then secured in place by hook and loop industrial-strength fasteners that remain in place until removed. The therapeutic hot and cold water belt is available in a variety of colors and is sized to fit any user. Materials preferably used include rubber for the pouch, elasticized material for the belt, hook and loop industrial-strength plastic for the fasteners, plastic for the screw and combed-cotton or mixed synthetic fibers for the pouch cover. The internal structure preferably is made out of the same material as the outside of the pouch.

This system is adjustably strapped in place and permits the wearer to move freely without movement of the belt and displacement of the pouch on the body part. It works naturally as a temperature therapy, and is portable.

Several advantages are derived from this arrangement. The belt offers comfort and relief from pain, such as by heat relieving muscle pain, by stimulating blood circulation, by relieving pains related to menstrual cramping in women and pain after childbirth, and by relieving back and muscle pain in general. It works as a natural temperature therapy. It can be applied about the waist, lower abdomen, back and other parts of the body, and then secured in place. It works to provide a hot, cold and warm temperature therapy treatment, with the user determining what temperature of water is to be used, and thereby poured into the pouch. The pouch retains its shape and resists ballooning, when water is contained in it. The belt can be sized for application to any part of the body or for use by an adult or child.

Other aims and advantages, as well as a more complete understanding of the present invention, will appear from the following explanation of an exemplary embodiment and the accompanying drawings thereof.

### BRIEF DESCRIPTION OF THE DRAWINGS

FIG. 1 illustrates a therapeutic hot and cold water belt and its covered water-containing pouch as embodied in the present invention and as placed about a wearer's waist or lower abdomen;

FIG. 2 is a front elevational view of the belt as depicted in FIG. 1, with its body-securing and fasteners disengaged with an open closure flap to show the funnel-shaped liquid fill port for filling the belt pouch and a plug for closing the fill port;

FIG. 3 is a front elevational view of the belt as illustrated in FIG. 2, with the flap closed over the liquid fill port after the plug has closed the fill port, and showing its fasteners engaged;

FIG. 4 is a back elevational view of the belt similar to that as viewed in FIG. 2, with the flap closed over the funnel-shaped fill port;

FIG. 5 is a view of an open cover for covering the pouch;

FIG. 6 is a view of the cover as depicted in FIG. 5, as placed over and closed about the pouch;

FIG. 7 is an enlarged view of the pouch portion of the belt, as shown in prior figures, specifically FIG. 2, with a portion of the pouch cut away to illustrate details of its funnel-shaped entry and fill port;

FIG. 8 is an enlarged view of the pouch portion of the belt, as shown in prior figures, specifically FIG. 2, with a portion of the pouch cut away to illustrate its interior ribs and interior water compartment;

FIGS. 9A, 9B and 9C are details of the inner supporting ribs of the pouch as shown in FIG. 8;

FIG. 10 is a cross-sectional view of the pouch taken along line 10-10 of FIG. 8, as filled with water, prior to being sealed by its plug, and disclosing its compartments as divided by its ribs;

FIG. 11 is a view of a portion of the pouch, as shown in FIG. 10, with its plug sealing the water entry port;

FIG. 12 is a partial view of the pouch, in its stored position, with the plug removed and the pouch inverted and hung upside down, to permit water to be drained from the pouch; and

FIGS. 13A and 13B respectively are views of the therapeutic hot and cold water belt pouch, in cross-section, as placed on a wearer, without the cover as depicted in FIGS. 5 and 6, illustrating its adaptability to being positioned on the wearer's abdomen (FIG. 13A) and lower back (FIG. 13B).

### DETAILED DESCRIPTION OF THE PREFERRED EMBODIMENTS

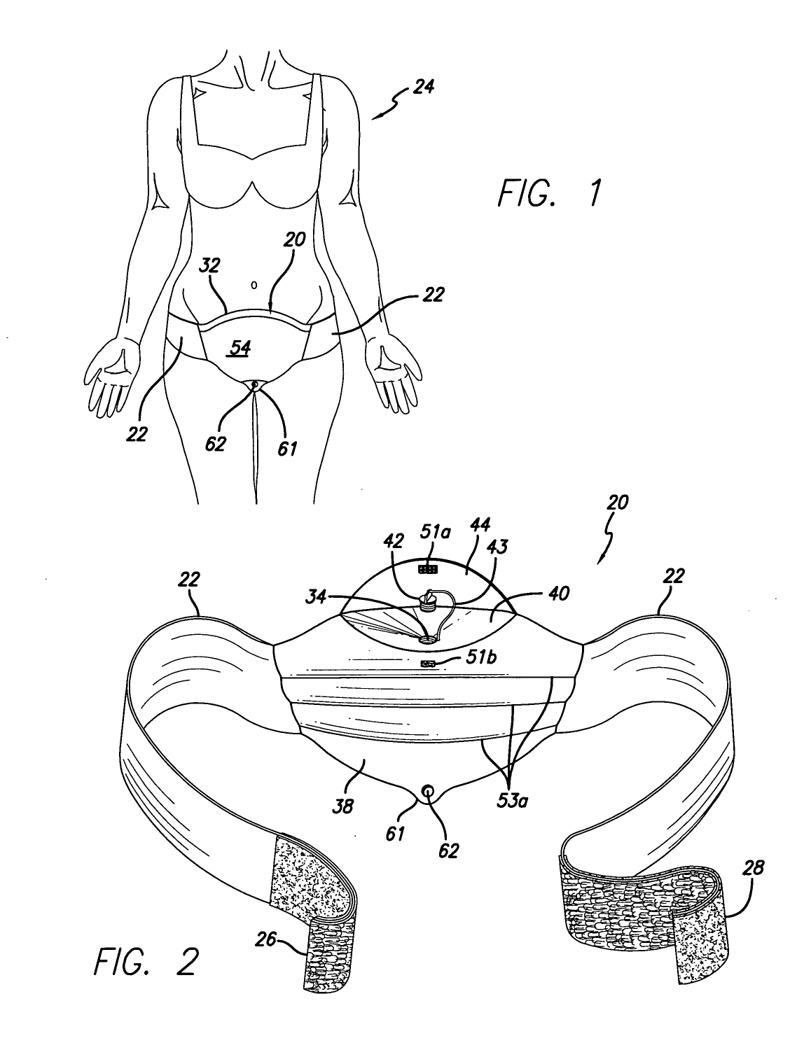

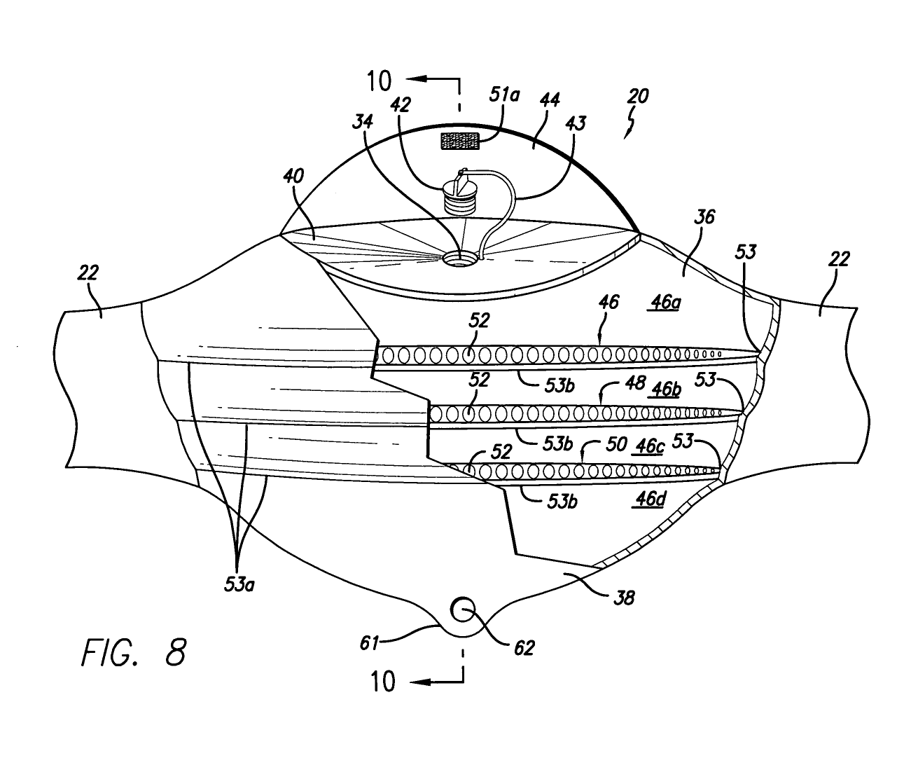

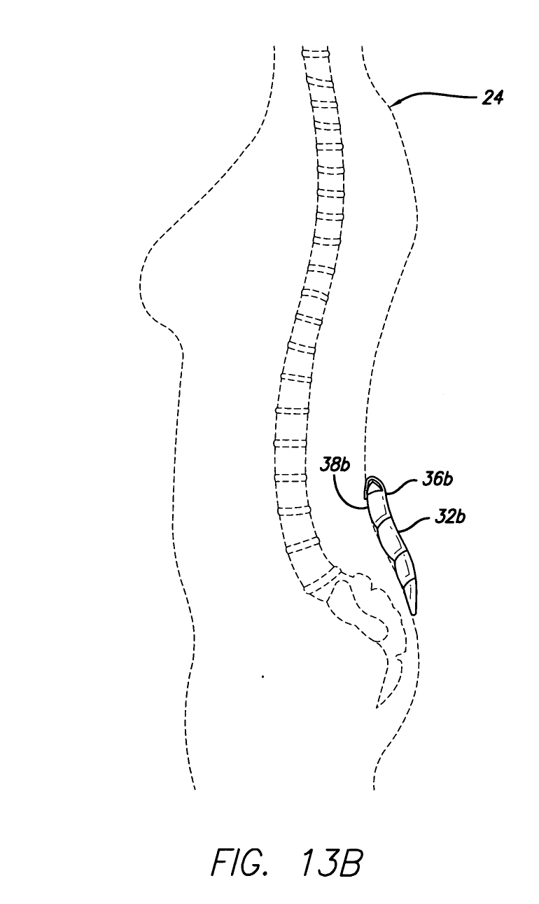

Accordingly, a therapeutic hot and cold water belt 20 comprises a band 22 of strong elastic material which is adapted to be placed about a wearer 24 and which is provided with ends 26 and 28 that are securable to one another for securing the band about the hips or other body part of the wearer. Band 22 may be constructed in any convenient manner, whether fully or partially elasticized. Ends 26 and 28 may utilize a hook-and-loop fastener system. Such a fastening system comprises two mating components, a hook member that has upstanding, hook-type fastener elements, and a loop member that has a surface that provides fibers or fiber loops with which the hook elements become engaged. Other equivalent fastening means may also be employed to advantage. In the present invention, the hook-and-loop fastener system utilized is double-sided, that is, one side of each end 26 and 28 will have hook-type fastener elements and the other side a loop-type fastener elements, to enable belt 20 to be reversibly attached to the person's body as desired. A water impermeable pouch 32, which is secured to band 22 between its ends, is adapted to hold water and has a threaded entry hole 34 (see FIGS. 2, 7, 8 and 10-12) for receiving and retaining the water. Hole 34 terminates a funnel-shaped entry 40 for aiding in the insertion of water into the pouch. Pouch 32 is preferably made of an elastomeric material, with its outside cover made with a mixture of combed cotton and synthetic fabric that will provide a smooth and comfortable feel to the wearer. Alternatively, the rubber pouch surface material itself can be coated with a mixture of combed cotton and synthetic materials. It is obvious, of course, that the producer of belt 20 can utilize any materials for manufacture of it or any of its constituent parts. As best seen in FIG. 8, the pouch comprises a pair of generally flat panels 36 and 38 which are secured together at their peripheries to form a generally flat pouch configuration. Alternately, the pouch may be molded in such a flat configuration. Because pouch 32 is intended to be placed on the person's body so that either panel 36 or 38 can be contacted with the skin (see FIGS. 13A and 13B), double-sided ends 26 and 28 enable such placement to occur.

A threaded plug 42 of hard plastic (see FIGS. 2, 7, 8 and 10-12) is adapted to thread into and seal entry 34 and to prevent the water from escaping from the pouch. Plug 42 is secured to the pouch adjacent to entry 34 in funnel 40 by a lanyard 43. The attachment of plug 42 to lanyard 43 is by a swivel joint to prevent twisting of the lanyard. As shown in FIGS. 2, 7, 8 and 10-12, a flap 44 is secured to pouch 32 adjacent to plug 42 and funnel 40 and is adapted to fold over and cover and protect the plug and entry 34 and to avoid, if desired, any direct contact with the user's body.

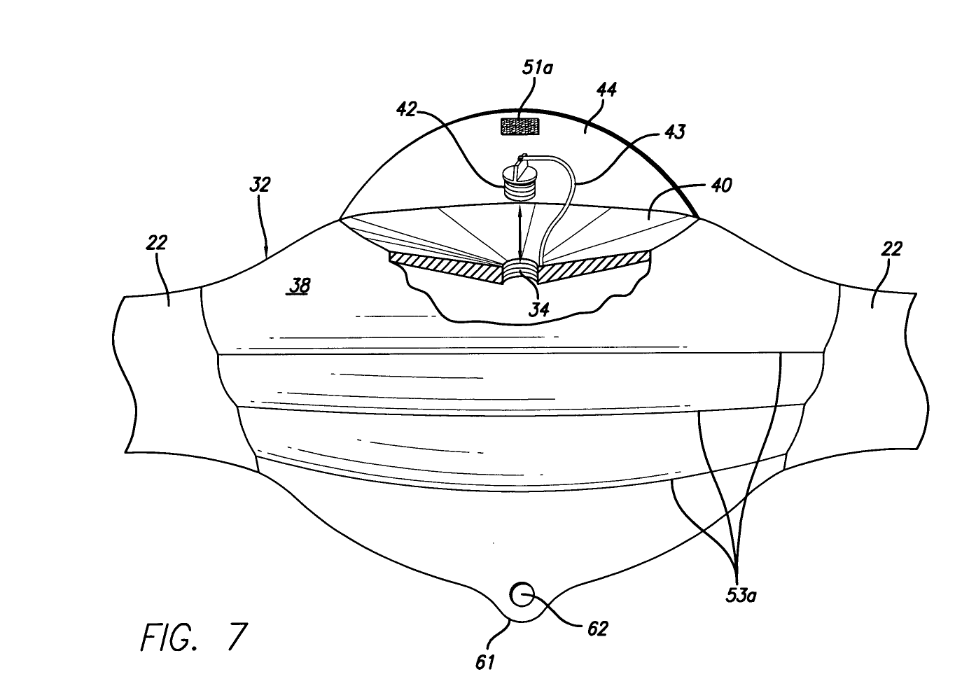

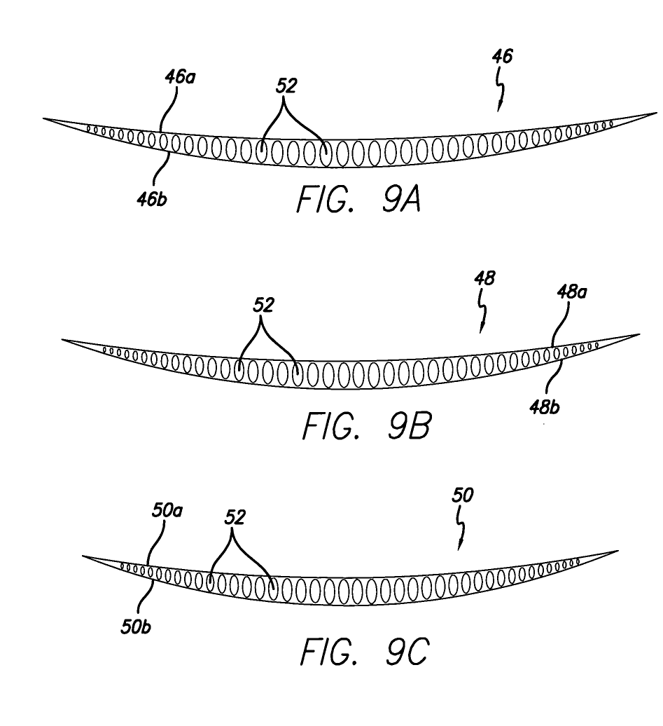

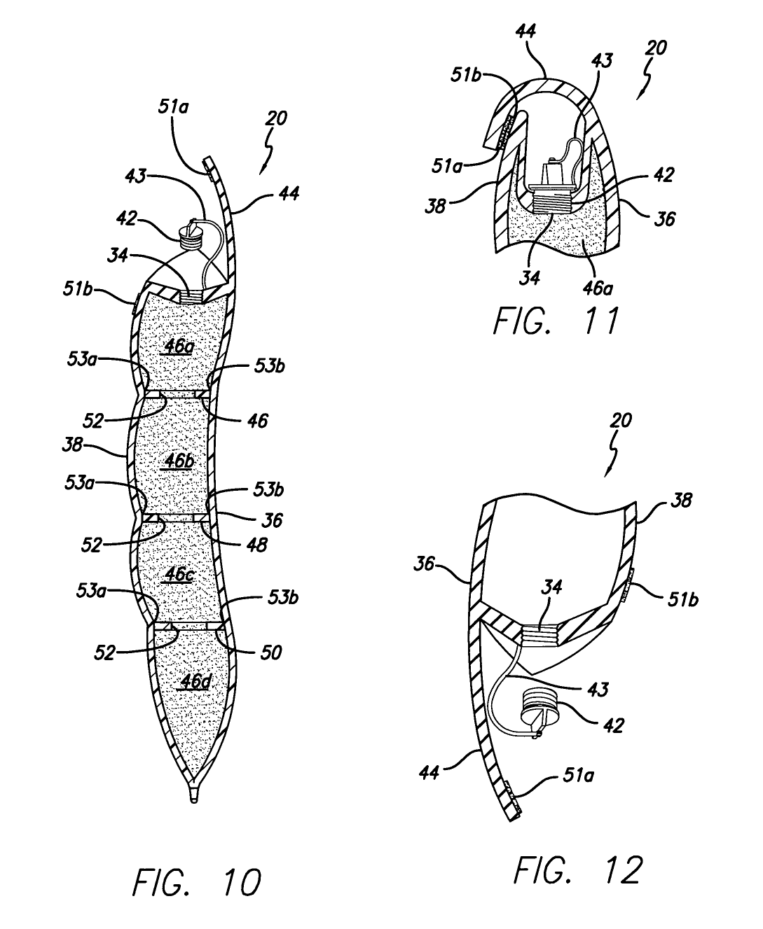

A rigidizing structure (see FIGS. 7, 8, and 9A-9C), comprising a plurality of parallelly placed ribs 46, 48 and 50, is formed within pouch 32 and provides the pouch with a shape which is compatible or moldable with that of the body part of the wearer. The back and front sides of the ribs, respectively rib backs 46a, 48a and 50a and rib fronts 46b, 48b and 50b, are respectively bonded to back and front panels 36 and 38 at bonds 53, 53a and 53b. The three ribs further divide the pouch into four horizontally oriented compartments 46a, 46b, 46c and 46d, as best seen in FIGS. 8 and 10. While three ribs and four compartments are disclosed, a greater or lesser number may be employed, as necessary to provide for the desired support for and size of the pouch. While placed in the parallel arrangement as shown, the ribs may be otherwise oriented than from side to side, but top to bottom or otherwise angled. Thus, for example, the compartmentalization may be vertically oriented rather than horizontally oriented, as depicted. The ribs have holes or openings 52 formed through their flat configurations for enabling water to flow through the ribs and thereby to completely flow into and fill the pouch and its compartments. The ribs are secured to the inner walls of the pouch interior, as depicted by bonds 53, 53a and 53b (FIGS. 8 and 10) not only to enable the pouch to retain its shape but also to prevent ballooning of the pouch when filled with water. Specifically, bond 53a defines the bond to panel 38 and bond 53b to panel 36. The ribs and their size and openings also control and limit the amount of water inserted into the pouch.

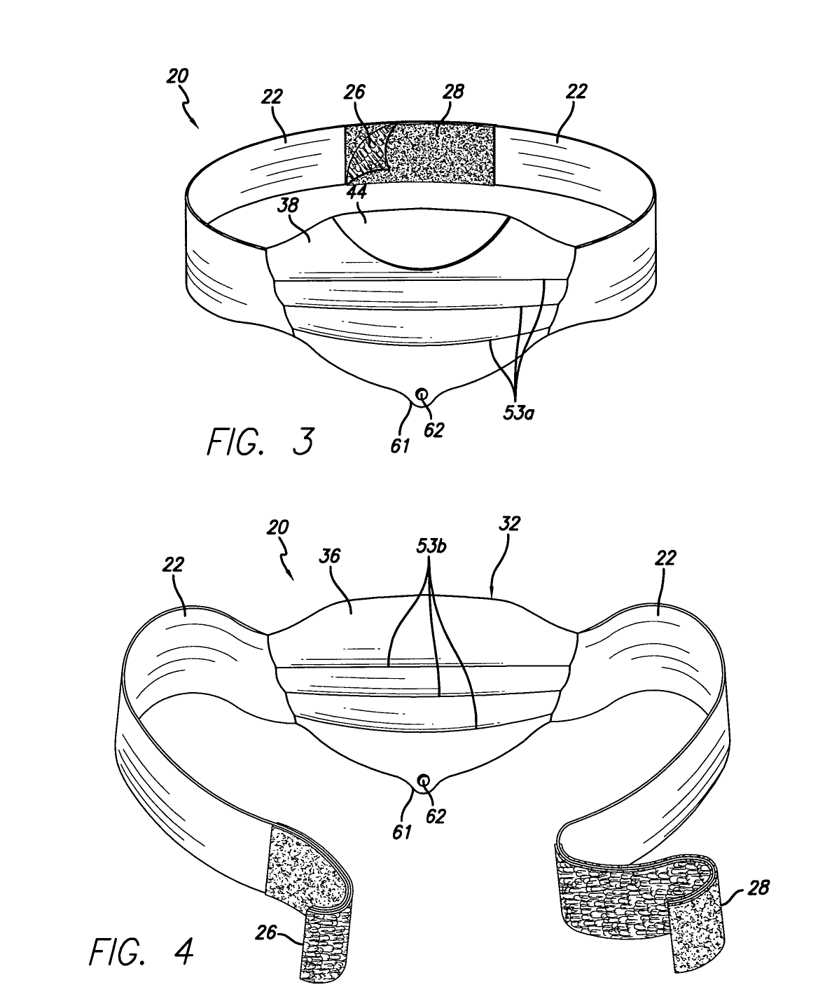

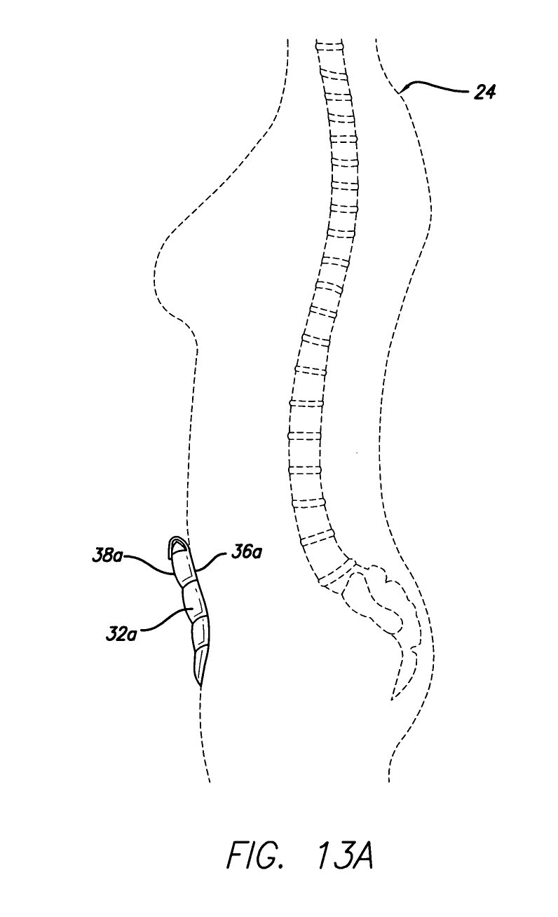

After pouch 32 is filled with water (as illustrated, for example, in FIGS. 2 and 10), and plug 42 is screwed into entry 34 to seal the pouch, flap 44 is folded over the sealed pouch entry and secured into place (as illustrated in FIGS. 3 and 11), such as by use of a hook-and-loop fastening or attachment system 51a and 51b. Alternatively, a snap-on fastener system may be used. As further depicted in FIG. 11, when part 51a is secured to part 51b of the hook-and-loop system (with plug 42 having been threadedly engaged in entry 34), the walls of funnel 40 surrounding threaded entry 34 are somewhat bunched up.

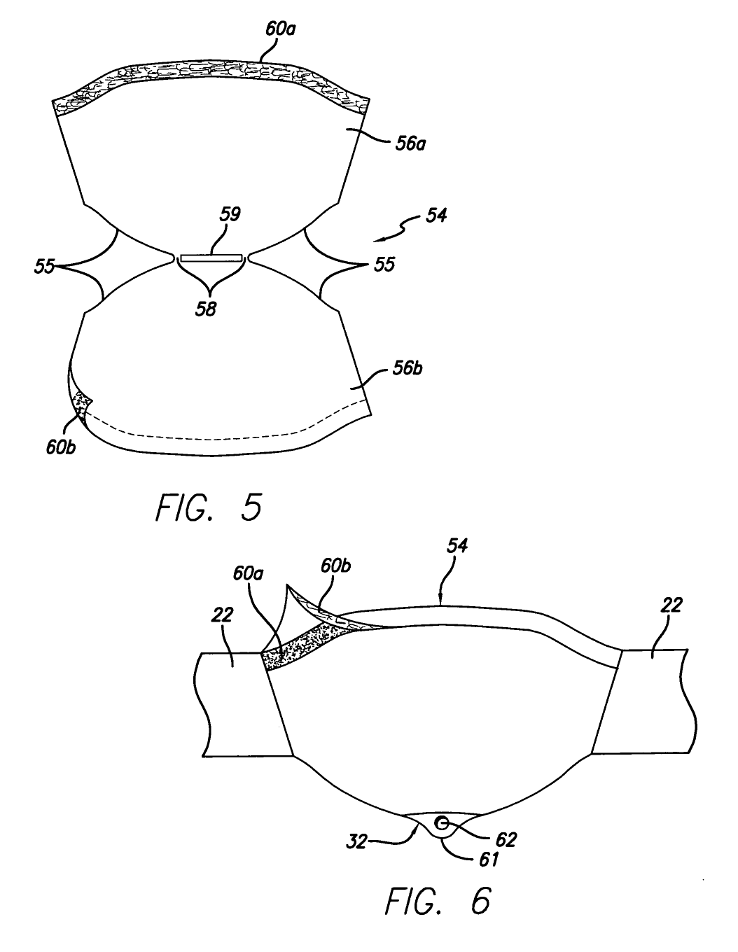

The assembly is completed by a cover 54 (as best seen in FIGS. 5 and 6) which comprises a pair of flat portions 56a and 56b of fabric (such as combed cotton or combed synthetic to provide comfort) which, in turn, are each generally configured as panels 36 and 38 of the pouch. Portions 56a and 56b are connected together at one of their respective ends such as by a connecting material 58, having an opening 59 for a projection 61 having an eyelet hole 62 (see FIGS. 1-4, 6, 7 and 10), as provided by the material of the portions so that they can act as a hinge. In addition, portions of the edges or seams adjacent to connecting material 58 (as denoted by indicium 55) may be sewn together to provide cover 54 with a pocket-like configuration and to add stability to it. The portions are fastenable together at their other ends 60a and 60b, such as by a hook-and-loop fastening or attachment system, so that end 60b can be folded over end 60a after having been placed, as shown in FIG. 6, about the pouch and secured thereabout by their hook-and-loop fastener system.

As shown in FIGS. 13A and 13B depending upon the part and configuration of the body onto which the water belt is to be applied, one or the other side of the pouch can be configured similarly as to that body part and placed in contact therewith. Accordingly, as depicted in FIG. 13A, a pouch 32a (illustrated in cross-section and without cover 54) is adapted to be placed on the wearer's abdomen so that its side 36a is pressed against the skin there. Alternately, as depicted in FIG. 13B, if the placement is desired for the wearer's lower back, a pouch 32b (depicted in cross-section and without cover 54) is adapted to be placed on the wearer's back so that its side 38b is pressed against the skin there. It is to be understood that, while the pouch as exemplified and as specifically configured in the drawings with respect to a person's body, it can be otherwise shaped so as to conform to any part of the body. For example, the portions as illustrated by panels 36 and 38 and additionally shaped by ribs 46, 48 and 50, may be curved or so made flexible, as to adapt the pouch to the body part.

When belt 20 is stored and not in use, as partially illustrated in FIG. 12, plug 42 is removed from entry 34, the belt and pouch 32 is invented, upside down, and drained of water, and hung up by hooking eyelet hole 62 to an eye-hook.

Although the invention has been described with respect to a particular embodiment thereof, it should be realized that various changes and modifications may be made therein without departing from the spirit and scope of the invention.

What is claimed is:

1. A therapeutic hot and cold water belt comprising:
a band which is adapted to be placed about a wearer and which is provided with ends that are securable to one another for securing said band about any one of several body parts of the wearer;
a water impermeable pouch which is secured to said band between said ends, which is adapted to hold water and which has an entry for receiving and retaining the water;
a seal adapted to seal said entry and to prevent the water from escaping said pouch;
a rigid structure formed within said pouch to provide said pouch with a shape which is compatible with that of the body part of the wearer.

2. A belt according to claim 1 wherein said rigid structure comprises a plurality of ribs spaced from one another.

3. A belt according to claim 2 wherein said ribs divide said pouch into a plurality of compartments.

4. A belt according to claim 3 further including openings in said ribs to facilitate filling of said compartments with water.

5. A belt according to claim 2 wherein said ribs divide said pouch into a plurality of horizontally oriented compartments.

6. A belt according to claim 2 wherein said pouch is provided with a given shape and said ribs are configured to fill out and maintain said shape, to resist ballooning of said pouch, and to limit and thereby conserve the quantity of water inserted into said pouch.

7. A belt according to claim 6 wherein said pouch comprises a pair of generally flat panels secured together at their peripheries to form a generally flat configuration and said ribs are secured between said panels in a generally parallel arrangement.

8. A belt according to claim 7 wherein said ribs are provided with openings opening through their flat configurations for separating said pouch into compartments and for enabling the water to reach entirely though said pouch.

9. A belt according to claim 7 wherein said pouch has differently configured front and back sides as defined by said panels which front and back sides are adaptable to contact similarly configured pars of the wearer's body.

10. A belt according to claim 1 further including a flap secured to said band adjacent to said seal and adapted to fold over and cover and protect said seal.

11. A belt according to claim 10 wherein said flap is formed integral with said band.

12. A belt according to claim 11 wherein said pouch entry comprises a threaded opening and said seal comprises a threaded plug for engagement with and threading into said threaded opening.

13. A belt according to claim 12 further comprising a lanyard secured to said plug and adjacent to said threaded opening.

14. A belt according to claim 1 further including an attachment mechanism secured to said band adjacent to said pouch for enabling said band to be hung when not secured about the body part of the wearer.

15. A belt according to claim 1 further including a cover for covering said pouch.

16. A belt according to claim 15 wherein said cover comprises a pair of flat portions, each generally configured as said pouch panels, said portions being hingedly secured to one another at one of their ends and having a securing mechanism at the other of their ends so as to enable said flat portions to embrace said pouch and to be secured to one another by said securing mechanism and to holdingly form a sandwich arrangement with said pouch.

17. A belt according to claim 16 wherein said cover is formed from a soft material so as to fit and feel comfortable against the body part of the wearer.

18. A belt according to claim 1 wherein said pouch has a generally flat body-conformable configuration including front and back paneled sides, further comprising:
a securing system enabling said ends to be secured together, said securing system including double-sided attachments for enabling alternate placement on any one of the body parts.

19. A belt according to claim 18 wherein said double-sided attachments comprise two mating components, a hook member that has upstanding, hook-type fastener elements, and a loop member that has a surface that provides fibers or fiber loops with which the hook elements become engaged, so that either one of said front and back paneled sides can be contacted with the body parts skin.

20. A container comprising:
a fluid impermeable pouch having an established shape and volume which is adapted to hold a fluid and which has an entry for receiving and retaining the fluid;
a seal adapted to seal said entry and to prevent the fluid from escaping from said pouch;
a rigid structure formed within said pouch to help maintain said pouch with its shape.

21. A container according to claim 20 wherein said rigid structure comprises a plurality of ribs spaced from one another.

22. A container according to claim 21 wherein said ribs divide said pouch into a plurality of compartments.

23. A container according to claim 22 further including openings in said ribs to facilitate filling of said compartments with water.

24. A container according to claim 21 wherein said ribs divide said pouch into a plurality of horizontally oriented compartments.
